(19) United States
(12) Patent Application Publication
(10) Pub. No.: US 2023/0192715 A1
(43) Pub. Date: Jun. 22, 2023

(54) MORPHINAN ISOMERS AND THEIR STRUCTURAL MODIFICATIONS AS NMDAR ANTAGONISTS AND NEUROPLASTOGENS

(71) Applicants: Paolo L. Manfredi, Miami Beach, FL (US); Charles E. Inturrisi, New York, NY (US); University of Padova, Padua (IT); Institute for Research in Biomedicine, Bellinzona (CH)

(72) Inventors: Paolo L. Manfredi, Miami Beach, FL (US); Charles E. Inturrisi, New York, NY (US); Andrea Mattarei, Perarolo di Vigonza (IT); Sara De Martin, Vigonza (IT); Jacopo Sgrignani, Almenno San Salvatore (IT)

(21) Appl. No.: 17/906,884

(22) PCT Filed: Mar. 24, 2021
(86) PCT No.: PCT/US21/23882

§ 371 Date: Sep. 21, 2022

Related U.S. Application Data
(60) Provisional application No. 62/993,805, filed on Mar. 24, 2020.

(51) Int. Cl. C07D 489/02; A61K 33/30; A61K 33/06; A61K 45/06

(52) U.S. Cl. CPC C07D 489/02; A61K 33/30; A61K 33/06; A61K 45/06

(57) ABSTRACT

Compounds, compositions, and methods for the treatment or prevention of symptoms, conditions, and diseases that may benefit from NMDAR modulation, including administering a compound of the morphinan family to a subject. The method may include administration of dextromorphine to a subject experiencing a symptom, condition, or disease that may benefit from NMDAR modulation, (except for the indications of pain and addiction), or may include administering dextrocodeine, dextrohydromorphone, dextrohydrocodone, dextrooxymorphone, dextrooxycodone, dextrooripavine, dextrothebaine, dextroethorphine, and dextrobuprenorphine to a subject experiencing a symptom, condition, or disease that may benefit from NMDAR modulation. Derivatives and stereoisomers of such compounds may also be used.

### CROSS-REFERENCE TO RELATED APPLICATION

This application claims the benefit of the filing date of U.S. Patent Application Ser. No. 62/993,805, filed on Mar. 24, 2020, the disclosure of which is incorporated by reference herein in its entirety.

### FIELD OF THE INVENTION

The present invention relates to the treatment of various symptoms, diseases, disorders, and conditions, and to compounds and/or compositions for such treatment.

### BACKGROUND OF THE INVENTION

The sections below are intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The present inventors have previously disclosed that select stereoisomers of molecules presently included in the opioid class modulate N-methyl-D-aspartate receptors (NMDARs) at doses and/or concentrations that do not result in clinically meaningful opioid adverse effects (narcotic effects), and that these select stereoisomers may be therapeutic and/or preventive for diseases, symptoms, and conditions.

For example, certain of the present inventors have previously disclosed that dextromethadone (and related stereoisomers and derivatives) can be used to treat the symptoms of pain and addiction (see U.S. Pat. No. 6,008,258) and can be used to treat select isolated psychological and/or psychiatric symptoms (see U.S. Pat. No. 9,468,611) at doses and/or concentrations that do not have clinically meaningful opioid receptor effects.

Additionally, the present inventors have also disclosed a platform of molecules, stereoisomers, and stereoisomer derivatives of molecules presently included in the opioid class, and a novel NMDAR in silico model developed ad hoc to select NMDAR modulators of the methadone family of compounds that could potentially have clinical uses.

In additional work in the methadone family of compounds, certain of the inventors have also disclosed that dextromethadone has rapid, robust, sustained, and statistically significant efficacy for major depressive disorder (MDD). This efficacy result is useful because MDD and treatment resistant depression (TRD) have been found to be linked to inflammatory states, cardiovascular disease and, in the brain, to neuronal loss and altered neuronal circuits. A double-blind placebo controlled prospective randomized clinical trial performed by certain of the inventors showed that dextromethadone can induce remission of disease (MADRS<10) in over 30% of patients compared to 5% in patients randomized to placebo, within the first week of treatment. Notably, the remission persisted for at least seven days after discontinuation of treatment, implying a disease modifying mechanism of action (e.g., modulation of neuroplasticity), rather than a symptomatic treatment. As opposed to a disease-modifying mechanism of action, the effects of purely symptomatic treatments for chronic conditions tend to decrease, cease, or may even rebound (worsening compared to pre-treatment baseline) at the discontinuation of therapy; clinical improvements, including improvement in symptoms, determined by disease modifying treatments tend instead to persist upon completion of the treatment cycle and discontinuation of the drug (e.g., immunotherapy for cancer, for multiple sclerosis or for rheumatoid arthritis). Also, symptomatic treatments tend to depend on receptor binding and sustained drug plasma levels while the effects of select disease modifying treatments may persist after plasma levels fall to negligible levels.

Additionally, certain of the present inventors have shown that dextromethadone can potentially modulate in vitro inflammatory biomarkers that are abnormal in neuropsychiatric diseases and conditions, including major depressive disorder (MDD) and treatment resistant depression (TRD), and in neurodegenerative diseases, such as dementias, including Alzheimer's disease, and in Parkinson disease and neurodevelopmental diseases, such as autism spectrum disorders, and other neuropsychiatric conditions such as schizophrenia and others.

While the above-described work has demonstrated various uses of dextromethadone (and other derivatives and members of the methadone family of compounds) for amelioration of pain, treatment of psychiatric symptoms, and modification of diseases and disorders, further such compounds would be useful. However, certain other compounds have not been considered to be useful for myriad reasons. For example, morphine, its isomers, and its derivatives have not been considered by those of ordinary skill in the art because the naturally occurring alkaloid in clinical use, levo-morphine, had been found to lack NMDAR activity (see, e.g., Gorman A L, Elliott K J, Inturrisi C E. The d-and l-isomers of methadone bind to the non-competitive site on the N-methyl-D-aspartate (NMDA) receptor in rat forebrain and spinal cord. Neurosci Lett. 1997 Feb. 14; 223(1):5-8. doi: 10.1016/s0304-3940(97)13391-2. PMID: 9058409) and therefore its isomer derivatives, for reasons related to stereoselectivity, or lack of thereof, were also expected by those of ordinary skill in the art to lack NMDAR modulating effects. This is because of the lack of stereoselectivity for NMDAR modulation seen with all other tested opioids: stereo-selectivity was not previously expected to significantly change NMDAR modulating activity for morphine stereoisomers, and thus these stereoisomers were thought to be inactive at the NMDAR and thus ineffective for diseases that could be improved by NMDAR blockers. Additionally, the structural group of morphine (structural group I) differs from the structural groups of drugs with known NMDAR actions (structural groups II and III from Codd E E, Shank R P, Schupsky J J, Raffa R B. Serotonin and norepinephrine uptake inhibiting activity of centrally acting analgesics: structural determinants and role in antinociception. J Pharmacol Exp Ther. 1995 September; 274(3):1263-70. PMID: 7562497). Furthermore, drugs in the Codd et al. II and III structural groups have additional actions at monoamine pathways (Codd et al., 1995; Rickli A, Liakoni E, Hoener M C, Liechti M E. Opioid-induced inhibition of the human 5-HT and noradrenaline transporters in vitro: link to clinical reports of serotonin syndrome. Br J Pharmacol. 2018; 175(3):532-543), while drugs in Codd structural group I lack these effects. These observations were also suggestive of a lack of NMDAR effects including other potentially therapeutic effects on neural plasticity for the drugs in structural group I, including theft stereoisomers.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Aspects of the present invention overcome the various drawbacks described above by providing compounds of the morphinan family (and compositions including same), including morphinan isomers (and structural derivatives thereof) as NMDAR antagonists and neuroplastogens. Methods for treatment or prevention of symptoms, diseases, disorders, and conditions that may involve NMDAR dysfunction, or which may benefit from NMDAR modulation, are also disclosed.

As described above, the present inventors have developed a novel NMDAR in silico model to select NMDAR modulators of the methadone family of compounds that could potentially have clinical uses. However, the present inventors have now updated this in silico model to identify further molecules with preventive and therapeutic potential in diseases and disorders caused or maintained by NMDAR dysregulation.

In particular, the present inventors have now unexpectedly determined that levomorphine has NMDAR predicted activity which, among hundreds of compounds tested, is the closest to the predicted activity for dextromethadone: these scores were 6.246 for dextromethadone and 6,136 for levomorphine (dextromethadone, as noted above, is a highly promising NMDAR modulator discovered by the current inventors that is currently in advanced clinical stages of development for neuropsychiatric and neurodegenerative diseases). Levomorphine is of the morphinan family of compounds, and dextromethadone is of the methadone family of compounds. The differences in structure among these two compound families (morphinan and methadone) are very marked—such that one of ordinary skill in the art would not consider morphinan compounds to have such NMDAR activity. As noted above, morphine, its isomers, and its derivatives have not been considered by those of ordinary skill in the art because the naturally occurring alkaloid in clinical use, levo-morphine, had been found to lack NMDAR activity (see, e.g., Gorman et al., 1997) and therefore its isomer derivatives, for reasons related to stereoselectivity, or lack of thereof, were also expected by those of ordinary skill in the art to lack NMDAR modulating effects, and were thought to be inactive at the NMDAR and thus ineffective for diseases that could be improved by NMDAR blockers.

The present inventors' finding regarding levomorphine unexpectedly signals potential NMDAR modulating activity for morphinan drugs in the Codd structural class I (and potential therapeutic and/or preventive effects similar to those previously disclosed by the inventors for dextromethadone). This is in contrast with previous findings by the inventors and others, and in particular in contrast with Gorman et al., 1997: in this paper the Ki for morphine was >100, in contrast with the Ki of 7.4 for dextromethadone.

And so, one aspect of the present invention is directed to a compound having a structure analogue to dextro-morphine and dextro-codeine according to formula I:

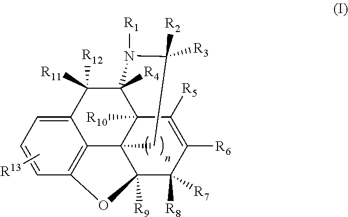

wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₃ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is comprised between 0 and 4.

Another aspect of the present invention is directed to a compound having a structure analogue to dextro-hydromorphone, dextro-hydrocodone, dextro-oxymorphone and dextro-oxycodone, according to formula II:

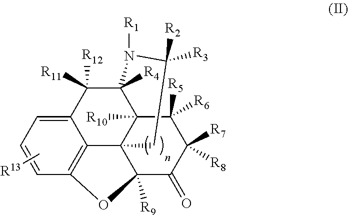

wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₃ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is comprised between 0 and 4.

Another aspect of the present invention is directed to a compound having a structure analogue to dextro-oripavine and dextro-thebaine, according to formula III:

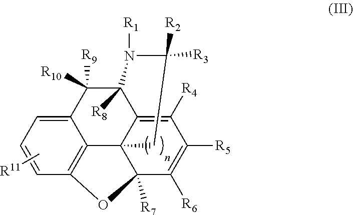

Wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₀ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₀ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₁ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is comprised between 0 and 4.

And another aspect of the present invention is directed to a compound having a structure analogue to dextro-ethorphine and dextro-buprenorphine, according to formula IV:

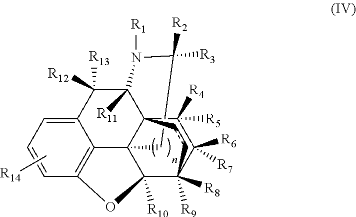

Wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₃ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₃ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₄ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
n is comprised between 0 and 4; and
the bridged bond between position 9 and 6 of the morphine scaffold is single or double.

Another aspect of the present invention is directed to a method for the treatment or prevention of symptoms, conditions and diseases that may benefit from NMDAR modulation, the method comprising administering a compound being dextromorphine to a subject experiencing a symptom, condition, or disease that may benefit from NMDAR modulation, except for the indications of pain and addiction. And another aspect of the present invention is directed to a method for the treatment or prevention of symptoms, conditions and diseases that may benefit from NMDAR modulation, the method comprising administering a compound chosen from dextrocodeine, dextrohydromorphone, dextrohydrocodone, dextrooxymorphone, dextrooxycodone, dextrooripavine, dextrothebaine, dextroethorphine, and dextrobuprenorphine to a subject experiencing a symptom, condition, or disease that may benefit from NMDAR modulation.

Another aspect of the present invention is directed to a method for the treatment or prevention of symptoms, conditions and diseases that may benefit from NMDAR modulation, the method comprising administering the compound of Formula I to a subject experiencing a symptom, condition, or disease that may benefit from NMDAR modulation, wherein the compound is a dextromorphine derivative, a dextrocodeine derivative, a stereoisomer of a dextromorphine derivative or a stereoisomer of a dextrocodeine derivative.

Another aspect of the present invention is directed to a method for the treatment or prevention of symptoms, conditions and diseases that may benefit from NMDAR modulation, the method comprising administering the compound of Formula II to a subject experiencing a symptom, condition, or disease that may benefit from NMDAR modulation, wherein the compound is a dextrohydromorphone derivative, a dextrohydrocodone derivative, a dextrooxymorphone derivative, a dextrooxycodone derivative, a stereoisomer of a dextrohydromorphone derivative, a stereoisomer of a dextrohydrocodone derivative, a stereoisomer of a dextrooxymorphone derivative, or a stereoisomer of a dextrooxycodone derivative.

Another aspect of the present invention is directed to a method for the treatment or prevention of symptoms, conditions and diseases that may benefit from NMDAR modulation, the method comprising administering the compound of Formula III to a subject experiencing a symptom, condition, or disease that may benefit from NMDAR modulation, wherein the compound is a dextrooripavine derivative, a dextrothebaine derivative, a stereoisomer of a dextrooripavine derivative or a stereoisomer of a dextrothebaine derivative.

Another aspect of the present invention is directed to a method for the treatment or prevention of symptoms, conditions and diseases that may benefit from NMDAR modulation, the method comprising administering the compound of Formula IV to a subject experiencing a symptom, condition, or disease that may benefit from NMDAR modulation, except for the indications of pain and addiction, wherein the compound is a dextroethorphine derivative, a dextrobuprenorphine derivative, a stereoisomer of a dextroethorphine derivative or a stereoisomer of a dextrobuprenorphine derivative.

These and other advantages of the application will be apparent to those of skill in the art with reference to the detailed description below.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Definitions

For the purposes of this disclosure, the present inventors define “diseases” as human and veterinary diseases, disorders, syndromes, and symptoms in their different stages, from preclinical stages to advanced stages, (including symptoms and signs of diseases, including prodromes and other manifestations of diseases).

For the purposes of this disclosure, the present inventors define “symptoms” as manifestations of diseases as defined above.

For the purposes of this disclosure, the present inventors define “treatment” as treatment and/or prevention, including primary and secondary prevention, and amelioration of conditions, symptoms, disorders, syndromes and diseases.

For the purposes of this disclosure, the present inventors define “conditions” as underperformance relative to the individual's potential and goals in cognitive, motor and social abilities and underperformance in special senses relative to the individual's potential and goals.

For the purposes of this disclosure, the present inventors define “functions” as functions of special senses (vision, olfaction, taste, hearing, and balance), including for improvement of vision.

For the purpose of this disclosure, the present inventors define “aging or ageing” as the accumulation of changes in a living being over time, leading to a deficit or deterioration in physical, psychological, and social abilities. The present inventors include in this definition accelerated aging and diseases due to physical and chemical factors, including environmental factors, toxins, and drugs, foods and lack of nutrients and vitamins and drug treatments.

For the purpose of this disclosure, the present inventors define “neural plasticity” as structural and functional changes in the nervous system occurring at any time during the life span starting from embryonic stages, including, in later stages of embryonic development: neuronal differentiation, neurogenesis; neuronal migration; modulation of a cell, including a neuron (soma or neurite) an astrocyte an oligodendrocyte, a microglial cell, its function, structure, size, shape and length; synaptic plasticity, including synaptogenesis, synaptic strengthening, synaptic weakening, spinogenesis, addition and/or loss of synaptic spines, “pruning”, changes in synaptic spine volume, changes in synaptic densities, including changes in specific synaptic proteins and pathways (including PD95, PD93,synapsin, GLUR1, including changes in mRNA coding for synaptic density protein and receptor subunits, including protein subunits of the AMPAR and of the NMDAR, and including modulation of the mTOR pathway and TrkB pathway and changes in neurotrophic factor pathways, including especially BDNF). Memory formation (synaptic strengthening) and memory clearing (synaptic weakening) are also structural and functional expressions of neural plasticity.

For the purpose of this disclosure, the present inventors define “neuroplastogen drugs” or “neuroplastogens” as drugs with the potential for modulating neural plasticity, as defined above. In order for a neuroplastogen drug to be potentially useful for prevention and/or treatment and/or diagnosis of diseases, disorders, symptoms, syndromes and conditions, at therapeutic dosages, the drug should be safe and well tolerated and the modulating action on neural plasticity should occur in the absence of clinically meaningful side effects, including in the absence of opioid receptor agonist cognitive negative side effects, in the absence of cognitive negative side effects induced by modulation of the NMDAR or 5-HT receptor pathways and/or narcotic and/or psychedelic/psychotomimetic side effects, including in the absence of “dissociative” symptoms.

For the purpose of this disclosure, the present inventors define “neuroplastogen dosage” and in particular “neuroplastogen dosage of a drug classified as an NMDAR antagonist and/or an opioid receptor modulator or a HT receptor modulator”, as a dose, dosage, posology or formulation, including modified release formulations, of a substance, (including a substance previously classified as an opioid or an opioid stereoisomers), with actions on neural plasticity and/or with modulating actions at the NMDAR and/or the 5-HT receptor, that can be administered at doses, dosages, posology and/or formulations, that do not cause clinically meaningful opioid or psychedelic/psychotomimetic effects and/or is well tolerated and safe. Neuroplastogen drugs may modulate NMDAR subunit transcription and synthesis and NMDAR assembly and expressio via downregulation of NMDAR Ca²⁺ currents and consequential downstream gene regulation, including regulation of genes that code for NMDAR subunits and other synaptic proteins, including structural synaptic and neurite proteins, and gene regulation for trophic factors, including BDNF, at dosages, posology and/or formulations that do not cause clinically meaningful off target effects, including without causing clinically meaningful opioid side effects relative to the indication including without clinically meaningful potential for addiction to opioids and without clinically meaningful respiratory depression, and/or without clinically meaningful negative alterations in consciousness, emotion, and cognition, including positive and negative psychotomimetic symptoms (psychedelic effects, psychedelic experience, psychotomimetic effects, dissociative effects). If the clinical indication for a neuroplastogen drug is a severe disease with poor prognosis, for example ALS, some opioid side effects and some psychotomimetic side effects may be acceptable, The mechanism of action of neuroplastogen drugs for their uses in the treatment of diseases and conditions proposed by the inventors consists in differential down regulation of excessive Ca²⁺ influx preferentially through hyperactive NMDARs (selective open channel block) subtypes A-D and particularly channels formed with subunits less or not sensitive to the Mg²⁺ block, e.g., NMDARs tonically and pathologically hyperactive, including those in particular containing 2C-D subunits, in select cells, cellular populations and/or cellular networks (in these select cells NMDARs may be hyperactive resulting in excessive Ca²⁺ influx) part of the nervous system or even in cells part of tissues, organs and systems outside of the nervous system (e.g., pancreas, liver, urogenital tract, bone, immune system, cardiovascular and respiratory system and platelets), as described by Du et al. 2016 (Du J, Li X H, Li Y J. Glutamate in peripheral organs: Biology and pharmacology, Eur J Pharmacol., 2016; 784: 42-48). The block of excessive Ca²⁺ influx reinstates cellular functions, including, for CNS cells, functions essential for physiological neural plasticity (e.g., mobilization and synthesis of synaptic proteins, including NMDAR subunits, and synthesis of neurotrophic factors, including BDNF) or other functions specific for the cells that had been affected by excessive Ca²⁺ influx (including cells outside of the CNS listed above (regulation of metabolic parameters and substances including blood sugar and insulin, metabolic and filtration activities, respiratory parameters, immune function, cell contractility and membrane potential, coagulation).

### DETAILED DESCRIPTION

Aspects of the present invention overcome the various drawbacks described above by providing compounds of the morphinan family (and compositions including same), including morphinan isomers (and structural derivatives thereof) as NMDAR antagonists and neuroplastogens. Methods for treatment or prevention of symptoms, diseases, disorders, and conditions that may involve NMDAR dysfunction, or which may benefit from NMDAR modulation, are also disclosed. Further aspects disclose the effectiveness and the potentially disease modifying effects of other select opioid stereoisomers and their derivatives, including the effects of morphinans, morphinan isomers and their derivatives, as signaled by the in silico NMDAR model developed ad hoc by the inventors, showing a matching activity for morphine and dextromethadone for a multiplicity of neuropsychiatric, metabolic and cardiovascular disorders.

Due to the unexpectedly matching NMDAR predicted effect for the morphine and methadone molecules in the present inventors' in silico model (initially presented in Intl. Patent Application No. PCT/US2019/055590 and updated since for better definition of the NMDAR blocking potential of select molecules, and on the grounds of the inventors prior work on the NMDAR effects of opioid and opioid isomers, and in light of the recently disclosed effects of dextromethadone, the present inventors now disclose that morphine and its stereoisomers and their derivatives and dextromorphine and its derivatives, and dextrohydromorphone and dextro-diacetyl-morphine, are potentially clinically useful for the treatment and/or preventions of symptoms, diseases and conditions triggered or maintained by NMDAR dysfunction. For dextromorphine, but not for other morphine stereoisomers and morphine derivatives, the present inventors exclude from the disclosed indication the treatment of pain and the treatment of addiction, indications already disclosed by others for dextromorphine (Stringer M, Makina M K, Milesa J, Morleya L S, d-Morphine, but not I-morphine, has low micromolar affinity for the non-competitive N-methyl-d-aspartate site in rat forebrain: Possible clinical implications for the management of neuropathic pain, Neuroscience Letters 295 (2000) 21-24; Wu H, Schwasinger E T, Terashvili M, Tseng L F, dextro-Morphine attenuates the morphine-produced conditioned place preference via the sigma 1 receptor activation in the rat, European Journal of Pharmacology 562 (2007) 221-226). There are no excluded indications for other morphine derivatives and their stereoisomers.

And so, the present invention overcomes the various drawbacks described above by providing compounds and compositions including morphinan isomers (and structural derivatives thereof) as NMDAR antagonists and neuroplastogens. In that regard, aspects of the present invention overcome the various drawbacks described above by providing compounds of the morphinan family (and compositions including same), including morphinan isomers (and structural derivatives thereof) as NMDAR antagonists and neuroplastogens. Methods for treatment or prevention of symptoms, diseases, disorders, and conditions that may involve NMDAR dysfunction, or which may benefit from NMDAR modulation, are also disclosed.

As described above, the present inventors have developed a novel NMDAR in silico model to select NMDAR modulators of the methadone family of compounds that could potentially have clinical uses. And, the present inventors have updated this in silico model to identify further molecules with preventive and therapeutic potential in diseases and disorders caused or maintained by NMDAR dysregulation.

In particular, in updating this in silico model, the present inventors unexpectedly determined that levomorphine has NMDAR predicted activity which, among hundreds of compounds tested, is the closest to the predicted activity for dextromethadone: these scores were 6.246 for dextromethadone and 6,136 for levomorphine (dextromethadone, as noted above, is a highly promising NMDAR modulator discovered by the current inventors that is currently in advanced clinical stages of development for neuropsychiatric and neurodegenerative diseases). Levomorphine is of the morphinan family of compounds, and dextromethadone is of the methadone family of compounds. The differences in structure among these two compound families (morphinan and methadone) are very marked—such that one of ordinary skill in the art would not consider morphinan compounds to have such NMDAR activity. The present inventors' finding regarding levomorphine unexpectedly signals potential NMDAR modulating activity for morphinan drugs in the Codd structural class I (and potential therapeutic and/or preventive effects similar to those previously disclosed by the inventors for dextromethadone). This is in contrast with previous findings by the inventors and others, and in particular in contrast with Gorman et al., 1997: in this paper the Ki for morphine was >100, in contrast with the Ki of 7.4 for dextromethadone.

And so, an aspect of the present invention is directed to a compound having a structure analogue to dextro-morphine and dextro-codeine according to formula I:

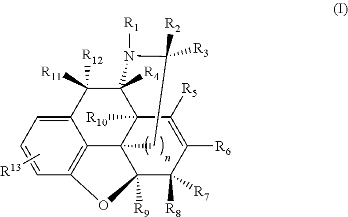

wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₃ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is comprised between 0 and 4.

Another aspect of the present invention is directed to compound having a structure analogue to dextro-hydromorphone, dextro-hydrocodone, dextro-oxymorphone and dextro-oxycodone, according to formula II:

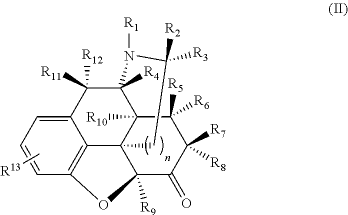

wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₃ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is comprised between 0 and 4.

Another aspect of the present invention is directed to a compound having a structure analogue to dextro-oripavine and dextro-thebaine, according to formula III:

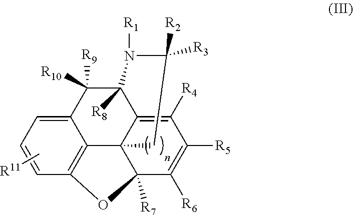

wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₀ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₀ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₁ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is comprised between 0 and 4.

And another aspect of the present invention is directed to compound having a structure analogue to dextro-ethorphine and dextro-buprenorphine, according to formula IV:

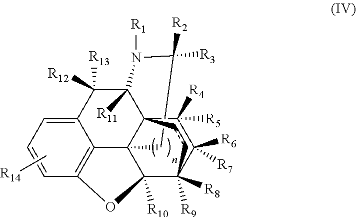

wherein R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₃ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₃ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₄ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
n is comprised between 0 and 4; and
the bridged bond between position 9 and 6 of the morphine scaffold is single or double.

Of note, the NMDAR actions of all the molecules in the opioid family and their stereoisomers previously studied and previously disclosed by the inventors (in U.S. Pat. No. 6,008,258; U.S. Pat. No. 9,468,611; International Patent Application No. PCT/US2018/016159; International Patent Application No. PCT/US2019/055590) were not thought to be stereoselective: e.g., for the previously disclosed molecules, racemate, dextro and levo isomers, all appeared to share similar NMDAR actions. The opioidergic actions were instead found to be stereoselective, allowing for the choice of the lesser opioidergic isomers for potential therapeutic uses of these molecules as NMDAR antagonists. In the present inventors' previous disclosures, the opioidergic actions, if any, of the lesser opioidergic stereoisomer were considered additional effects (off target effects, side effects) of the intended primary action, which is modulation of the NMDARs.

In the present inventors' previous disclosures, several opioid and opioid derivatives were disclosed as potential NMDAR modulators for the treatment and/or prevention of a multiplicity of diseases, symptoms and conditions. However, the present inventors had not disclosed morphine, its isomers and its derivatives (nor did those of ordinary skill in the art consider morphine, its isomers, and its derivatives useful) because the naturally occurring alkaloid in clinical use, levo-morphine, had been found to lack NMDAR activity (see, e.g., Gorman et al., 1997) and therefore its isomer derivatives, for reasons related to stereoselectivity, or lack of thereof (as detailed above) were also expected by those of ordinary skill in the art to lack NMDAR modulating effects. This is because of the lack of stereoselectivity for this effect (NMDAR modulation) seen with all other tested opioids: stereo-selectivity was not previously expected to significantly change NMDAR modulating activity for morphine stereoisomers, and thus these stereoisomers were thought to be inactive at the NMDAR and ineffective for diseases that could be improved by NMDAR blockers. Additionally, the structural group of morphine (structural group I) differed from the structural groups of drugs with known NMDAR actions (structural groups II and III from Codd et al., 1995). Furthermore, drugs in the Codd et al. II and III structural groups have additional actions at monoamine pathways (Codd et al., 1995; Rickli et al., 2018), while drugs in Codd structural group I lack these effects. These observations were also suggestive of a lack of NMDAR effects including other potentially therapeutic effects on neural plasticity for the drugs in structural group I, including their stereoisomers.

Now, based on additional and unexpected evidence developed by the present inventors and as disclosed herein, the inventors disclose that dextromorphine, while belonging to a different structural group (group I) is a unique molecule among opioid stereoisomers (as compared to other opioid stereoisomers with known NMDAR antagonist actions) because it has stereoselective NMDAR antagonist actions (a novel and unexpected finding of the present inventors). Furthermore, dextromorphine does not have monoamine actions, another distinguishing unique feature among opioid stereoisomers with NMDAR antagonist action (insofar as opioids and their stereoisomers with NMDAR blocking actions also have monoamine action at transporters (Codd et al. 1995) or at receptors (Rickli et al., 2018) (another novel and unexpected finding). These unique features for morphinans: (1) NMDAR stereoselectivity, (2) different structural group, (3) lack of 5-HT pathway actions, and (4) extremely low affinity and/or negligible affinity for opioid receptors for certain isomers, is advantageous for the treatment and/or prevention of select symptoms, diseases and conditions or for the treatment of select patients that benefit from NMDAR antagonistic actions in the absence of activity at opioid receptors or in the absence of activity at 5-HT pathways.

The naturally occurring morphine alkaloid in widespread clinical use for the treatment of pain, is isolated from the juice of the opium poppy (Papaver somniferum), and is stereochemically identified as a levorotatory form: levo isomer; (−)-morphine, levo-morphine. Levo-morphine is recognized as a potent mu opioid agonist with no action as a NE or 5-HT re-uptake inhibitor (Codd et al., 1995), no actions at serotonin receptors (Rickli et al, 2018), and no actions at the NMDAR (Gorman et al., 1997). Dextromorphine has no opioid agonist action and no actions as a NE or 5-HT re-uptake inhibitor (Codd et al., 1995), but has been found to act at the NMDAR (Stringer et al., 2000). And now, according to the present inventors' in silico model of the NMDARs, dextromorphine has now been shown by the present inventors to have actions at NMDARs that are very close to those exerted by dextromethadone. Recently, dextromethadone has been found by the inventors to be a clinically effective NMDAR modulator and diseases modifying treatment for patients with MDD.

Opioid receptors and NMDAR coexist in the same areas of the brain (Narita M, Hashimoto K, Amano T, et al., Post-synaptic action of morphine on glutamatergic neuronal transmission related to the descending antinociceptive pathway in the rat thalamus, J Neurochem. 2008; 104(2): 469-478. doi:10.1111/j.1471-4159.2007.05059.x) and are associated in post-synaptic structures of neurons (Rodríguez-Muñoz M, Sánchez-Blázquez P, Vicente-Sánchez A, Berrocoso E, Garzón J. The mu-opioid receptor and the NMDA receptor associate in PAG neurons: implications in pain control, Neuropsychopharmacology. 2012; 37(2):338-349. doi:10.1038/npp.2011.155). Opioid agonists, including levomorphine, increase Ca²⁺ currents when binding to mu opioid receptors (this effect on Ca²⁺ currents is blocked by a mu antagonist) (Narita et al., 2008). When the mu antagonist is present, morphine becomes a negative allosteric modulator (Ca²⁺ are decreased, similarly to the actions of an open channel blocker) (Narita et al.), Positive allosteric modulators (PAMs) act via two main mechanisms, (a) increasing the maximal response to glutamate and/or (b) shifting the ED₅₀ of glutamate to the left (Hackos O H, Hanson J E, Diverse models of NMDA receptor positive allosteric modulation: Mechanisms and consequences, Neuropharmacology, 2017, 112 (Pt A), 34-35). Toxic PAMs may be exogenous substances or endogenous substances such as inflammatory mediators or their intermediates. Based on Narita et al., 2008 and Hackos et al., 2016, levomorphine is an aPAM. Testing with differential concentrations of glutamate could also uncover a bPAM mechanism for levomorphine. The coupling of a drug with agonist actions at the mu opioid receptor with a mu opioid antagonist, as disclosed by certain of the present inventors in International Patent Application No. PCT/US2018/016159, may thus change the effect of the drug on Ca²⁺ currents from an enhancer (aPAM and/or bPAM) to a blocker (NAM). However, for diseases where the select targeting of NMDARs structurally associated with opioid receptors may be crucial (e.g., MDD and related disorders), a dual action drug (i.e., a MOR-agonist/NMDAR-antagonist) may work better. Additionally, for the treatment of pain, in order to abolish the NMDAR activation by opioid agonists, the combination of an agonist, (e.g., levomorphine) with an NMDAR antagonist, (e.g., dextromorphine) may be more effective than either isomer alone. When considering the combination of an agonist (e.g., levomorphine) with an NMDAR antagonist (e.g., dextromorphine) for the treatment of pain, racemorphine may be a more effective drug (less tolerance, less addiction potential: both tolerance and addiction are caused by NMDAR hyperactivation in neurons with opioid receptors). Racemorphine would be produced by adding the synthetic dextromorphine to the natural levomorphine. This formulation would be produced by varying the enantiomeric excess to achieve the best pharmacological match.

Without being bound to any theory, the present inventors hypothesize that the efficacy of low affinity NMDAR antagonists effective for MDD and related disorders may be improved when the NMDAR antagonist also has affinity for opioid receptors: this affinity for opioid receptors will allow selective targeting of the hyperactive NMDARs associated with opioid receptors (endorphin pathway). This explains why memantine, an effective NMDAR blocker with no affinity for opioid receptors does not work for MDD and why naloxone abolishes the antidepressant effects of ketamine: by binding to opioid receptors it interferes with the binding at the same site of ketamine and therefore interferes with its action at NMDARs.

MDD may thus be a disease of the endorphin pathway where NMDAR structurally associated to opioid receptors have become pathologically hyper-stimulated: endorphins cannot bind effectively to opioid receptors because their associated NMDARs are hyperactive. NMDAR blockers, selective for NMDAR structurally associated with opioid receptors, allow endorphins to bind to opioid receptors, allowing physiological mood regulation to resume.

Unexpectedly, the present inventors' novel in silico model of the NMDAR revealed that morphine exerts NMDAR predicted activity that matches dextromethadone. In light of the present inventors' in silico work, the present inventors re-visited tests performed by Stringer M, Makina M K, Milesa J, Morieya L S, d-Morphine, but not l-morphine, has low micromolar affinity for the non-competitive N-methyl-d-aspartate site in rat forebrain: Possible clinical implications for the management of neuropathic pain, Neuroscience Letters 295 (2000) pp. 21-24, which confirm the NMDAR modulating actions of dextromorphine but not levomorphine (levomorphine's lack of NMDAR antagonistic activity was also shown by Gorman et al., 1997), and without actions on NE and 5-HT (as had been shown by Codd et al., 1995). In light of the present inventors' work on opioids, their isomers and NMMDARs, and their effects on BDNF and m-ToR (De Martin S. Vitolo O, Bernstein C, Alimonti A, Traversa S, Inturrisi C E, Manfredi P L, The NMDAR Antagonist Dextromethadone Increases Plasma BDNF Levels in Healthy Volunteers Undergoing a 14-Day In-Patient Phase 1 Study, ACNP 57th Annual Meeting: Poster Session II. ACNP 57th Annual Meeting: Poster Session II. Neuropsychopharmacol. 43, 228-382 2018) and new studies showing induction of mRNA for NMDAR1 and induction of select NMDAR subunit proteins by dextromethadone (Intl. Patent Application No. PCT/US2019/055590), and in light of the present inventors' recent proof-of-concept phase 2 study results with dextromethadone for the treatment of MDD (which unexpectedly suggested robust, rapid and sustained disease modifying effects), the present inventors now disclose the potential uses of dextromorphine and select morphinans for the prevention and/or treatment of diseases, symptoms, and conditions triggered or maintained by NMDAR hyperactivity including diseases, symptoms and conditions disclosed in the present inventors' previous applications for dextromethadone and other opioid isomers and other structurally modified opioids (with the exception of the treatment of pain, already disclosed by Stringer et al., 2000, and with the exception of the treatment of addiction, already disclosed by Wu et al. 2007, and by Tseng in U.S. Patent Application Publication No. 2008/0200370).

Furthermore, the NMDAR action of dextromorphine is not accompanied by inhibitory actions on re-uptake of NE and 5-HT (Codd et al., 1995) and is not accompanied by serotoninergic actions or action at DAT (Rickli et al., 2018). This lack of actions at monoamine pathways may offer advantages over other opioids and opioid stereoisomers with NMDAR activity and inhibitory NET and SERT actions and/or additional serotoninergic activity, including dextromethadone, for the treatment of select symptoms, diseases, and conditions or for the treatment of select patients, e.g., for patients for whom modulation or activation of serotonin pathways is contraindicated.

Furthermore, the morphinan molecules disclosed herein are potentially effective for indications where NMDAR modulating actions are desired in the absence of actions on monoaminergic pathways, e.g., NE and 5-HT and DA pathways, for example, for uses in the treatment of MDD in patients already taking drugs that act on the adrenergic, serotoninergic, and dopaminergic systems, and for whom additional monoamine modulatory effects are not needed or contraindicated [as in the case of some patients taking MAOIs, SSRI, SNRI, or other antidepressant or antipsychotics with actions on monoamine pathways, including atypical antidepressants, antipsychotics, for example when used as adjunctive treatment for patients partially refractory to other treatments (e.g., TRD) or in select patients with MDD where the pathogenesis of the disorder is unrelated or unresponsive to modulation of the monoamine pathways, or in other clinical indications where interference with the monoamine systems may be contraindicated, such as in patients dementia and agitation, patients with ADHD, patients with Rett syndrome, patients with suicidal ideation, patients with hypertension and/or CAD, patients with cardiac arrythmias or patients genetically or pharmacologically predisposed to cardiac arrythmias (e.g., long QT syndrome etc.) and patients at risk for the serotoninergic syndrome].

In addition, the sigma 1 actions of dextromorphine, combined with its NMDAR modulating actions and lack of monoaminergic actions, may be particularly effective for a multiplicity of neuro-psychiatric disorders and symptoms where this receptor may be involved, such as agitation, including agitation in neurodevelopmental diseases, e.g., autism spectrum disorders, and including agitation in neurodegenerative diseases, including dementias, pseudobulbar affect, select MDD patients, schizophrenia, etc. One of the inventors has successfully tested morphine for the treatment of agitation in dementia (Manfredi P L, Breuer B, Wallenstein S, Stegmann M, Bottomley G and Libow L. Opioid Treatment for Agitation in Patients with Advanced Dementia. Int J Ger Psy 2003; 18:694-699). In that study, the treatment with morphine was aimed at treating pain—with the assumption that any agitation was caused by pain in those subjects suffering from dementia (and so, treating pain with morphine would have the effect of resolving the agitation, due to the analgesic effect of morphine on the pain). In light of the work on sigma 1 receptor (Wu H, Schwasinger E T, Terashvili M, Tseng L F, dextro-Morphine attenuates the morphine-produced conditioned place preference via the sigma1 receptor activation in the rat, European Journal of Pharmacology 562 (2007) 221-226) and NMDAR (Stringer et al., 2000), and the present inventors' in silico NMDAR model showing effects matching dextromethadone and in light of the newly discovered disease modifying effects of dextromethadone (described above), the successful treatment of agitation demented patients as studied by Manfredi et al., 2003, may not be secondary to analgesic actions, as hypothesized by Manfredi et al., 2003, but may be secondary to actions at the sigma receptor, NMDAR, or other receptors not primarily involved in analgesia, corroborating the present application for select morphine isomer and morphine derivatives, including dextromorphine and its derivatives, including dextrohydromorphone, as sigma 1 and NMDAR modulators for the prevention and/or treatment of diseases, symptoms, and/or conditions potentially treated or prevented by NMDAR and/or sigma receptor modulation.

Hydromorphone is a hydrogenated ketone of morphine. The chemical modification of the morphine molecule to hydromorphone results in higher lipid solubility and greater ability to cross the blood brain barrier (BBB) that potentially produces rapid and complete central nervous system penetration and is likely to have differential penetration in select brain areas compared to morphine and in the case of dextrohydromorphone potential differential penetration into CNS cells and potential differential binding to receptors, including NMDARs, including NMDAR subtypes, including differential actions at NMDARs that are part of super-complexes and may more difficult to reach by hydrophilic compounds, and including differential actions on different brain areas and circuits. Due to its higher lipid solubility and greater BBB penetration dextrohydromorphone may therefore be a more effective NMDAR blocker and sigma 1 agonist compared to dextromorphine for select patients or symptoms and/or diseases and/or conditions.

The present inventors therefore also disclose dextrohydromorphone and dextro-diacetyl-morphine and other morphine derivatives for all for the diseases, disorders, symptoms, conditions, and manifestations thereof disclosed in International Patent Application Nos. PCT/US2019/055590 and PCT/US2018/016159 (the diseases, disorders, symptoms, conditions, and manifestations thereof disclosed in those applications being incorporated by reference herein in theft entireties), without the exceptions listed for dextromorphine, as dextrohydromorphone and dextro-diacetyl-morphine and other disclosed morphine derivatives have not been previously disclosed as potential therapeutic agents.

Examples of such diseases, symptoms, and conditions may include Alzheimer's disease; presenile dementia; senile dementia; vascular dementia; Lewy body dementia; cognitive impairment [including mild cognitive impairment (MCI) associated with aging and with chronic disease and its treatment], Parkinson's disease and Parkinsonian related disorders, including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; epilepsy; NS trauma; NS infections; NS inflammation [including inflammation from autoimmune disorders (such as NMDAR encephalitis), and cytopathology from toxins (including microbial toxins, heavy metals, pesticides, etc.)]; stroke; multiple sclerosis; Huntington's disease; mitochondrial disorders; Fragile X syndrome; Angelman syndrome; hereditary ataxias; neuro-otological and eye movement disorders; neurodegenerative diseases of the retina like glaucoma, diabetic retinopathy, and age-related macular degeneration; amyotrophic lateral sclerosis; tardive dyskinesias; hyperkinetic disorders; attention deficit hyperactivity disorder (“ADHD”) and attention deficit disorders; restless leg syndrome; Tourette's syndrome; schizophrenia; autism spectrum disorders; tuberous sclerosis; Rett syndrome; Prader Willi syndrome; cerebral palsy; disorders of the reward system including but not limited to eating disorders [including anorexia nervosa (“AN”), bulimia nervosa (“BN”), and binge eating disorder (“BED”)], trichotillomania; dermotillomania; nail biting; substance and alcohol abuse and dependence; migraine; fibromyalgia; depression; PTSD; anxiety disorders including SAD; RLS; temporary cognitive impairment; emotional lability in pseudobulbar palsy; treatment resistant depression; Rett syndrome; major depressive disorder; and peripheral neuropathy of any etiology.

In addition to the diseases and their symptoms and manifestations as outlined above, the aspects of the present invention also relate to the treatment and/or prevention of metabolic-endocrine diseases including the metabolic syndrome and increased blood pressure, high blood sugar, excess body fat including liver fat, and abnormal cholesterol and/or triglyceride levels, type 2 diabetes and obesity, and diseases of the eye, including optic nerve diseases, retinal diseases, vitreal diseases, corneal diseases, glaucoma and dry eye syndrome.

Some examples of neurological symptoms and manifestations associated with these and other disorders may include: (1) a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; (2) abnormal movements, including akathisia, bradykinesia, tics, myoclonus, dyskinesias (including dyskinesias relate to Huntington's disease, levodopa-induced dyskinesias and neuroleptic-induced dyskinesias), dystonias, tremors (including essential tremor), and restless leg syndrome; (3) parasomnias, insomnia, and disturbed sleep pattern; (4) psychosis; (5) delirium; (6) agitation; (7) headache; (8) motor weakness; spasticity; impaired physical endurance; (9) sensory impairment (including impairment and loss of vision and visual field defects, impairment and loss of sense of smell, taste and hearing) and dysesthesias; (10) dysautonomia; and/or (11) ataxia, impairment of balance or coordination, tinnitus, and neuro-otological and eye movement impairments.

In addition to any neurological symptoms or manifestations, any cognitive dysfunction in an individual may be secondary to a neurodevelopmental or neurodegenerative disease such as Alzheimer's disease or Parkinson's disease and Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; or may be caused by diseases where the cognitive decline is multifactorial and in part related to treatment of another disease, such as may be seen in cancer, renal failure, epilepsy, HIV, use of therapeutic and recreational drugs, and aging/senescence of cells. Brain radiation therapy and electroconvulsive treatment are examples of therapies potentially associated with cognitive dysfunction.

The absolute configuration of the morphinan skeleton of (−)-sinomenine is enantiomeric to natural (−)-morphine. Straightforward synthetic manipulation of (−)-sinomenine allows for the obtainment of dextromorphine and the disclosed congeners as described by Iijima et al., 1978 (Iijima I, Minamikawa J, Jacobson A E, Brossi A, Rice K C, Studies in the (+)-morphinan series. 5. Synthesis and biological properties of (+)-naloxone, J Med Chem. 1978 April; 21(4):398-400).

The present inventors now disclose that derivatives with more hydrophilic properties (e.g., dextromorphine) may be more indicated for the treatment diseases and conditions secondary to NMDAR dysfunction in peripheral tissues and cells (Du et al., 2016; Kalev-Zylinska M L, Green T N, Morel-Kopp M C, et al. N-methyl-D-aspartate receptors amplify activation and aggregation of human platelets. Thromb Res. 2014; 133(5):837-847. doi:10.1016/j.thromres.2014.02.011) (e.g., pancreatic, liver, lung, bone, urogenital, cardiac, blood and connective tissues, Langerhans cells, hepatocytes, macrophages, osteoblasts and osteoclasts, urogenital cells and lymphocytes and platelets) for the treatment of metabolic, respiratory, cardiovascular, urogenital (including infertility and premature ovarian failure), bone, blood (including lymphocytic disorders and immune disorders and coagulation disorders, including DIC) and cancer and connective tissue disorders, including fibromyalgia. Lipophilic derivatives (e.g., dextrohydromorphone) may be more indicated for the treatment of neuropsychiatric disorders, especially disorders where sigma 1 receptor actions and NMDAR actions are desired in the absence of monoamine pathway activation, e.g., for the treatment of MDD and dementia, including the treatment of agitation associated with dementia and all neuropsychiatric indications where blood brain barrier penetration may be favorable to the effects.

The present inventors' in silica model of the NMDAR has shown that morphine has activity which is the closest to dextromethadone when compared to hundreds of molecules structurally related to opioids: the in silico scores were 6.246 for dextromethadone and 6,136 for levomorphine (dextromethadone, as noted above, is a highly promising NMDAR modulator discovered by the current inventors that is currently in advanced clinical stages of development for neuropsychiatric and neurodegenerative diseases). The present inventors now tested the compounds of Formulae I-IV (above) in this unique in silico model and the results are shown below in Table 1, with these results signaling NMDAR antagonistic activity at the pore channel for these compounds.

To accomplish this, the present inventors proceeded with (1) the testing of these molecules in silico in order to determine their potential for NMDAR channel block and (2) the synthesis of select molecules. This will be followed by (3) more advanced and specific in vitro and in vivo tests for NMDAR activity, including electro-physiologic testing of NMDARs to characterize relative affinity (methods for which are described in International Patent Application No. PCT/US2018/016159, those methods being incorporated by reference herein) and (4) confirming mechanism of block suggested by the in silico testing (e.g., drugs with uncompetitive type block actions are likely to be safer and more effective because of their selective actions at sites of NMDAR dysfunction and not at sites with physiologic activities). The present inventors have already begun verifying excitotoxicity protection in vitro and are evaluating select morphinans for safety and activity in in vitro experimental models. Finally, after entering into the clinical phases of development, the present inventors will confirm tolerability and effectiveness in human trials, first in healthy volunteers, and then in patients with specific diseases and conditions.

Molecular Modeling Investigations of Morphinans Binding to the Trans-Membrane Site of the NMDA Receptor GluN1-GluN2A-B-C-D Tetramer Subtypes in Their Closed State

Until recently, because of technical limitations in both expression and purification of the trans-membrane proteins of the NMDAR, the structure of the trans-membrane domain of NMDAR had not been characterized at an atomistic level. In 2014, Gouaux and co-workers solved the structure of the Xenopus laevis GluN1-GluN2B NMDA receptor by X-ray crystallography (Lee C H, Lü W, Michel J C, Goehring A, Du J, Song X, Gouaux E. NMDA receptor structures reveal subunit arrangement and pore architecture. Nature. 2014 Jul. 10; 511(7508):191-7. doi: 10.1038/nature13548. Epub 2014 Jun. 22. PMID: 25008524; PMCID: PMC4263351.). This structure was obtained in the presence of Ro25-6981, a partial agonist, and MK-801, an ion channel blocker, and represents a closed state of the NMDAR. Given the high similarity of this structure with the human sequence, the present inventors used the structure identified by Protein Data Bank (PDB) code 4TLM as the starting point for their computational studies. The present inventors investigated (a) dextromethadone, an established NMDAR antagonist, currently in clinical development for several indications; (b) positive controls (ketamine, memantine, dextromethorphan, amantadine, MK-801, PCP) all known NMDA open channel blockers acting at the PCP site at the trans-membrane domain with known affinities and known clinical effects; the first four drugs are in clinical uses while PCP is a schedule I drug with no clinical uses and MK-801 is a high affinity antagonist with severe side effects that impede its clinical use; and (c) morphinans . The docking and energy scores were found to be in a similar range as those of established NMDAR channel blockers.

Table 1 shows docking results for morphinans.

While NMDAR antagonists acting at the trans-membrane domain of the receptor currently in clinical use are thought to exert their effects by binding to the open NMDAR, for the purpose of this computational model, the present inventors studied the binding to the closed conformation of the channel: clinically effective NMDAR antagonist drugs also bind to the PCP site in the closed state (Zanos P, Moaddel R, Morris P J, Riggs L M, Highland J N, Georgiou P, Pereira E F R, Albuquerque E X, Thomas C J, Zarate C A Jr, Gould T D, Ketamine and Ketamine Metabolite Pharmacology: Insights into Therapeutic Mechanisms, Pharmacol Rev. 2018 July; 70(3):621-660) and their “trapping” index in the closed state, a reflection of the relation of “onset” and “offset” time of action, can be an indication of clinical tolerability and effectiveness (Zanos et al., 2018; Huei-Sheng Vincent Chen and Stuart A. Lipton. The chemical biology of clinically tolerated NMDA receptor antagonists. Journal of Neurochemistry, 2006, 97, 1611-1626). Effective NMDAR modulators should therefore bind the open channel but also briefly (e.g., for a few milliseconds) bind the closed channel (“foot in the door” concept), while avoiding prolonged “trapping”. In the closed state, in the presence of Mg²⁺, 2A and 2B NMDAR subtypes are impermeable to Ca²⁺ and therefore channel blockers may more effectively block Ca²⁺ currents in the 2C and 2D subtypes that maintain permeability to Ca²⁺ currents in the closed state (Kuner T, Schoepfer R. Multiple structural elements determine subunit specificity of Mg²⁺ block in NMDA receptor channels. J Neurosci. 1996; 16(11):3549-3558; Kotermanski S E, Johnson J W. Mg²⁺ imparts NMDA receptor subtype selectivity to the Alzheimer's drug memantine, J Neurosci. 2009; 29(9):2774-2779; Hansen K B, Yi F, Perszyk R E, et al. Structure, function, and allosteric modulation of NMDA receptors. J Gen Physiol. 2018; 150(8):1081-1105. doi:10.1085/jgp.201812032). Furthermore, in docking calculations, the ligand is built inside the hosting binding site and therefore the closed conformation is more apt to evaluate the ligand/site interaction: the trajectory of the ligand to the binding site is not considered by the docking calculation.

While the prior computational NMDAR subtype built for this in silico testing was the GluN1-GluN2B tetramer composed by 2 GluN1 subunits and 2 GluN2B subunits, the current in silico model provides information on all NMDAR subtypes, including NMDARs containing 2C and 2D subunits, which as detailed above, are more likely to be the in vivo targets for NMDAR channel blockers tested by the in silico model. As detailed in prior disclosure from the applicants, among other discoveries, the applicants discovered that dextromethadone increases levels of PD95, GluR1 in vivo and that in vitro dextromethadone increases mRNA for NMDAR1, offering additional insight in the neural plasticity potential of dextromethadone and other NMDAR channel blockers that are an object of this disclosure.

To improve the computational efficiency of the present inventors' calculations, only the trans-membrane region of the receptor, where the PCP binding site is located, and where the FDA-approved and clinically tolerated NMDA antagonists also act (dextromethorphan, ketamine/esketamine, memantine), and where the present inventors postulate the morphinans, (that are an object of the present disclosure) act, was included into the simulated models. The goal of this computational portion of the inventors' work is to define the potential of select molecules for blocking the NMDAR pore with measurable parameters, similar to dextromethadone, that may be key for their binding to the trans-membrane domain of the NMDAR in order to achieve a block of the pore channel that is precisely modeled to prevent or treat select diseases. Each morphinan, aside from having unique onset/offset/trapping and unique actions on NMDAR subtypes and variances as described in the application, which may be advantageous for select diseases, will also have unique actions at other receptors, including affinity or lack of affinity for other receptors, and among those with affinity for other receptors, unique effects, ranging from agonist to inverse agonist effects, and unique PK characteristics, which may also offer benefits for prevention and treatment of select diseases and disorders.

Receptor Preparation

First, the receptor was prepared by the ‘protein preparation wizard’ procedure available in the Schrödinger suite, from Schrödinger of New York, N.Y. (https://www.schrodinger.com/) for molecular modelling.

This procedure automatically assigns the correct protonation state, completes missing side chains or small region, and assigns the correct name to the atoms. Then considering the data available in the Orientations of Proteins in Membranes database (OPM) database, (Lomize M A, Pogozheva I D, Joo H, Mosberg H I, Lomize A L. OPM database and PPM web server: resources for positioning of proteins in membranes. Nucleic Acids Res. 2012 January; 40(Database issue):D370-6. doi: 10.1093/nar/gkr703. Epub 2011 Sep. 2. PMID: 21890895; PMCID: PMC3245162) the receptor model was immersed in a membrane model formed by 1-palmitoyl-2-oleoyl-sn-glycero-3-phosphocholine (POPC) molecules.

Docking of Known Drugs

The first attempt to dock the molecules to investigate inside the receptor conformation directly derived from x-ray studies was done using the Glide software (available from Schrödinger, of New York, N.Y.) (https://www.schrodinger.com/glide). During the docking procedure the protein region in which the drug can be positioned was manually defined.

In this case, because of the lacking of a co-crystallized ligand, this region was defined considering the residues identified critical for the memantine binding by Dougherty and co-workers (Limapichat W, Yu W Y, Branigan E, Lester H A, Dougherty D A. Key binding interactions for memantine in the NMDA receptor. ACS Chem Neurosci. 2013 Feb. 20; 4(2):255-60. doi: 10.1021/cn300180a. Epub 2012 Dec. 7. PMID: 23421676; PMCID: PMC3751542).

The present inventors reduced the van der Waals (VdW) radii of the molecules in order to permit a more ‘flexible’ adaptation of the molecules to the receptor.

The docking calculations for levopropoxyphene were successful and produced a model of the drug receptor complex, while confirming the potential NMDAR blocking activity of levopropoxyphene.

Then, in order to permit the creation of a site more suitable for drug binding, the levopropoxyphene-receptor complex immersed into the membrane was simulated for 250 ns by Molecular Dynamics (MD) simulations.

The final conformation of the receptor was then used to perform new docking calculations with the same set-up applied initially.

The calculations were successful and structures of the drug receptor complex were obtained for the drugs tested.

Molecular Dynamic (MD) Simulations of the Receptor Drug Complexes

The systems composed by the drug, the receptor, and the membrane were then simulated by MD for 1 μsec. The present inventors produced trajectories for the complexes with: dextromethadone, memantine, ketamine, dextromethorphan, PCP, MK-801, and the morphinan molecules that are an object of this disclosures.

Virtual Pre-Screening

The receptor model used to dock dextromethadone, memantine, ketamine, dextromethorphan, PCP, MK-801 was then used to screen the morphinans. For this purpose, the 2D chemical structures of the molecules were transformed in 3D models for which all the possible protonation states were calculated. All the ligands were docked inside the receptor and their affinity scored by GlideScore—a specific scoring function for drug-protein interactions, based on the Glide software of Schrödinger, of New York, N.Y. (https://www.schrodinger.com/glide) and shown in Table 1

Effective binding events are always characterized by a negative difference in free energy (Delta G) between the bound and the unbound state (i.e. the free energy of the complex is lower than that calculated for the isolated ligand and target).

In the present inventors' calculations, several molecules—including dextromethadone—were predicted to have a negative Delta G value. In particular, ligand/receptor binding affinity—expressed by Delta G values—of many of the new compounds tested was similar or more negative of the value obtained for ketamine, a drug known for its activity at the NMDAR with FDA approved clinical indications for anesthesia. The more negative Delta G values, with respect to ketamine and other reference molecules, obtained for the different compounds tested on the developed protein model, suggest potential differences in drug receptor interactions, different onset/offset and trapping values, and a more effective binding affinity with consequential different clinical effects, which ultimately may be better suited for one or more diseases. As the experimentation with morphinans advances, the present inventors are likely to be able to characterize each new molecule with unique PD and PK parameters which may prove advantageous for select diseases and conditions.

As mentioned above in order to select the new compounds for undergoing synthesis and testing in excitotoxicity protection models in vitro the present inventors developed a new in silico NMDAR model and performed a preliminary validation study with MK-801 (control), dextromethadone, and the morphinans. The newly designed and tested in silico morphinans are now undergoing synthesis and further testing in vitro before planning in vivo and clinical experimental trials.

TABLE 1
Results for certain compounds of Formulae I-IV, signaling NMDAR
antagonist activity at the pore channel

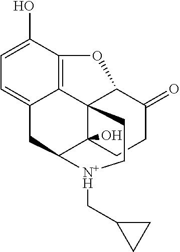

title: d-naltrexone
glide gscore: −7.853

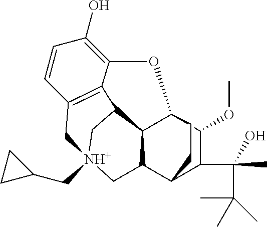

title: d-buprenorphine
glide gscore: −7.481

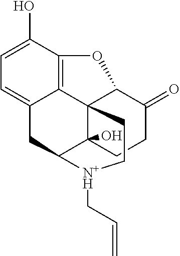

title: d-naloxone
glide gscore: −7.463

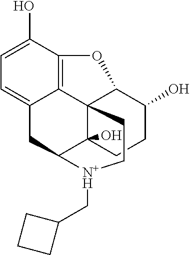

title: d-nalbuphine
glide gscore: −7.428

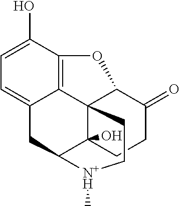

title: d-oxymorphone
glide gscore: −7.326

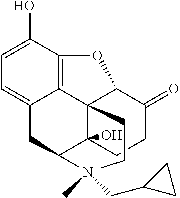

title: d-methylnaltrexone
glide gscore: −7.317

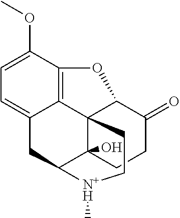

title: d-oxycodone
glide gscore: −7.275

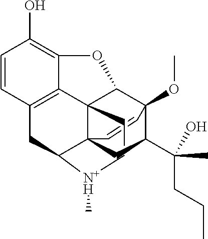

title: d-ethorphine
glide gscore: −7.123

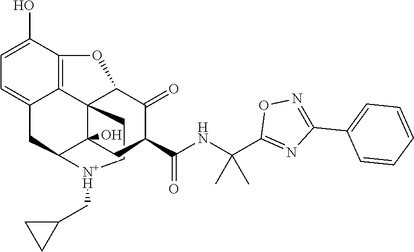

title: d-naldemedine
glide gscore: −6.997

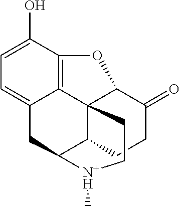

title: d-hydromorphone
glide gscore: −6.845

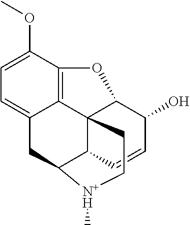

title: d-codeine
glide gscore: −6.616

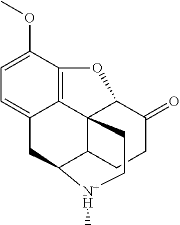

title: d-hydrocodone
glide gscore: −6.566

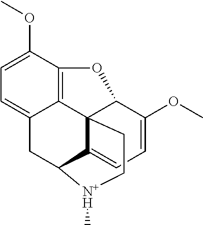

title: d-thebaine
glide gscore: −6.498

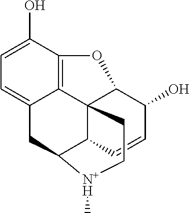

title: d-Morphine
glide gscore: −6.436

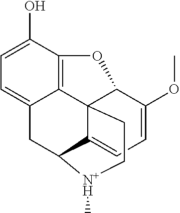

title: d-oripavine
glide gscore: −6.291

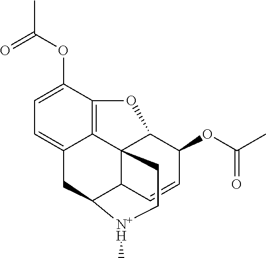

title: d-heroine
glide gscore: −6.784

As noted, the present inventors' in silico model of the NMDAR has shown that morphine has activity which is the closest to dextromethadone when compared to hundreds of molecules structurally related to opioids: the in silico scores were 6.246 for dextromethadone and 6,136 for levomorphine as is shown in Table 2, below (dextromethadone, as noted above, is a highly promising NMDAR modulator discovered by the current inventors that is currently in advanced clinical stages of development for neuropsychiatric and neurodegenerative diseases.

TABLE 2

| 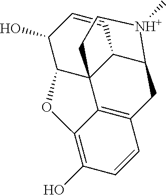 [SMILES: C[N@H+]1CC[C@@]23c4c5ccc(O)c4O[C@@H]2[C@H](O)C=C[C@@H]3[C@@H]1C5] | |
|---|---|
| title | Levomorphine |
| glide gscore | −6.246 |
| 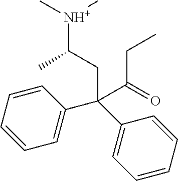 [SMILES: CCC(=O)C(C[C@H](C)[NH+](C)C)(c1ccccc1)c1ccccc1] | |
| title | Dextromethadone |
| glide gscore | −6.136 |

And so, an aspect of the present invention is directed to a method for the treatment or prevention of symptoms, conditions and diseases that may benefit from NMDAR modulation, the method comprising administering a compound being dextromorphine to a subject experiencing a symptom, condition, or disease that may benefit from NMDAR modulation, except for the indications of pain and addiction. And another aspect of the present invention is directed to a method for the treatment or prevention of symptoms, conditions and diseases that may benefit from NMDAR modulation, the method comprising administering a compound chosen from dextrocodeine, dextrohydromorphone, dextrohydrocodone, dextrooxymorphone, dextrooxycodone, dextrooripavine, dextrothebaine, dextroethorphine, and dextrobuprenorphine to a subject experiencing a symptom, condition, or disease that may benefit from NMDAR modulation.

In these methods, the compound may be formulated as a modified release, long-acting preparation. Further, in these methods, the administering of the compound may be performed orally, buccally, sublingually, rectally, vaginally, nasally, via aerosol, transdermally, parenterally, epidurally, intrathecally, intraauricularly, intraocularly, or topically, including eye drops and other ophthalmic formulations, and including iontophoresis and dermatologic formulations.

Further, in these methods, the administration of the compound may occur in conjunction with the administering of another substance. For example, administration of the compound may occur in combination with administering an antidepressant. Such an antidepressant may include typical or atypical antidepressants. For example, a typical antidepressant may include (but not be limited to) an SSRI or an SNERI, whereas an atypical antidepressant may include (but not be limited to) bupropion.

As another example, administration of the compound may occur in combination with administering an antipsychotic (including atypical antipsychotics).

As another example, administration of the compound may occur in combination with administering a drug that modulates a serotonin receptor. In one embodiment, the serotonin receptor may be a 5-HT2A agonist.

And, as yet another example, administration of the compound may occur in combination with administering magnesium or zinc.

Another aspect of the present invention is directed to a method for the treatment or prevention of symptoms, conditions and diseases that may benefit from NMDAR modulation, the method comprising administering the compound of Formula I to a subject experiencing a symptom, condition, or disease that may benefit from NMDAR modulation, wherein the compound is a dextromorphine derivative, a dextrocodeine derivative, a stereoisomer of a dextromorphine derivative or a stereoisomer of a dextrocodeine derivative.

Another aspect of the present invention is directed to a method for the treatment or prevention of symptoms, conditions and diseases that may benefit from NMDAR modulation, the method comprising administering the compound of Formula II to a subject experiencing a symptom, condition, or disease that may benefit from NMDAR modulation, wherein the compound is a dextrohydromorphone derivative, a dextrohydrocodone derivative, a dextrooxymorphone derivative, a dextrooxycodone derivative, a stereoisomer of a dextrohydromorphone derivative, a stereoisomer of a dextrohydrocodone derivative, a stereoisomer of a dextrooxymorphone derivative, or a stereoisomer of a dextrooxycodone derivative.

Another aspect of the present invention is directed to a method for the treatment or prevention of symptoms, conditions and diseases that may benefit from NMDAR modulation, the method comprising administering the compound of Formula III to a subject experiencing a symptom, condition, or disease that may benefit from NMDAR modulation, wherein the compound is a dextrooripavine derivative, a dextrothebaine derivative, a stereoisomer of a dextrooripavine derivative or a stereoisomer of a dextrothebaine derivative.

Another aspect of the present invention is directed to a method for the treatment or prevention of symptoms, conditions and diseases that may benefit from NMDAR modulation, the method comprising administering the compound of Formula IV to a subject experiencing a symptom, condition, or disease that may benefit from NMDAR modulation, except for the indications of pain and addiction, wherein the compound is a dextroethorphine derivative, a dextrobuprenorphine derivative, a stereoisomer of a dextroethorphine derivative or a stereoisomer of a dextrobuprenorphine derivative.

In the methods of these aspects (i.e., administering compounds of one of Formulae I-IV), the compound may be formulated as a modified release, long-acting preparation. Further, in these methods, the administering of the compound may be performed orally, buccally, sublingually, rectally, vaginally, nasally, via aerosol, transdermally, parenterally, epidurally, intrathecally, intraauricularly, intraocularly, or topically, including eye drops and other ophthalmic formulations, and including iontophoresis and dermatologic formulations.

Further, in these methods, the administration of the compound may occur in conjunction with the administering of another substance. For example, administration of the compound may occur in combination with administering an antidepressant. Such an antidepressenat may include typical or atypical antidepressants. For example, a typical antidepressant may include (but not be limited to) an SSRI or an SNERI, whereas an atypical antidepressant may include (but not be limited to) bupropion.

As another example, administration of the compound may occur in combination with administering an antipsychotic (including atypical antipsychotics).

As another example, administration of the compound may occur in combination with administering a drug that modulates a serotonin receptor. In one embodiment, the serotonin receptor may be a 5-HT2A agonist.

And, as yet another example, administration of the compound may occur in combination with administering magnesium or zinc.

As noted above, certain of the present inventors have recently discovered the disease-modifying potential for dextromethadone, a methadone isomer with NMDAR blocking actions without clinically meaningful opioid actions. Among other findings suggestive of disease modifying effects (neuroplasticity effects and effects on biomarkers), the inventors disclose that patients with MDD unresponsive to at least one adequate trial with an antidepressant can remit (MADRS<10) after only 1 week of treatment with dextromethadone and that this remission is sustained after discontinuation of therapy. The fact that the remission persists after discontinuation of treatment suggest that the action of dextromethadone is not purely symptomatic but is potentially determined by neural plasticity mechanisms previously disclosed by the inventors, e.g., mechanisms related to the expression of new NMDAR channels and/or production of BDNF. The inventors had previously signaled the neural plasticity effects of dextromethadone in vitro and in vivo and had shown that dextromethadone increases BDNF in humans (De Martin S, Vitolo O V, Bernstein G, Alimonti A, Traversa S, Inturrisi C E, Manfredi O L, The NMDAR Antagonist Dextromethadone Increases Plasma BDNF levels in Healthy Volunteers Undergoing a 14-day In-Patient Phase 1 Study. ACNP annual meeting, Dec. 9-13, 2018; Hollywood, Fla.).

The present inventors now disclose that certain morphinan compounds, including dextromorphine and its derivatives, including dextrohydromorphone, including dextro-diacetyl morphine, including their fluoro-derivatives and including their nitro-derivatives and their fluoro-nitro-derivatives, including their deuterated forms and the deuterated forms of their derivatives are potentially effective for diseases disorders symptoms and conditions for which NMDAR modulating actions may be therapeutic. For dextromorphine (but not for its derivarives and deuterated forms) the present inventors exclude the indications of addiction and pain.

The scientific rationale for including deuterated forms is outlined by the inventors in International Patent Application No. PCT/US2018/016159. In that regard, as noted above, experimental and clinical evidence presented, analyzed, and interpreted by the inventors supports the use of d-methadone for many clinical indications. One of the experimental studies analyzed by the inventors in International Patent Application No. PCT/US2018/016159 suggests that deuterium incorporation increases the NMDA antagonistic affinity of d-methadone. And because changes in NMDAR antagonistic activity may change the clinical effects of the compound, the inventors now disclose that deuterated forms of morphinan compounds, and the deuterated forms of their derivatives, are potentially effective for diseases disorders symptoms and conditions for which NMDAR modulating actions may be therapeutic.

The scientific rationale for fluoro and for nitro derivatives is outlined by the inventors in Intl. Patent Application No. PCT/US2019/055590. As noted therein, fluoro-derivatives, nitro-derivatives and fluoro-nitro-derivatives and deuterated fluoro-derivatives, deuterated nitro-derivatives and deuterated fluoro-nitro-derivatives are of particular interest for optimization of structure activity relationship with the NMDAR, because of the potential for improving PK parameters (especially for fluoro-derivatives) and because of additional NMDAR modulating mechanisms and prevention of reactive nitrogen species cellular damage (especially for nitro-derivatives). In view of this, the inventors now disclose that morphinan compounds, including their fluoro-derivatives, their nitro-derivatives, and their fluoro-nitro-derivatives, are potentially effective for diseases disorders symptoms and conditions for which NMDAR modulating actions may be therapeutic.

While the present invention has been disclosed by reference to the details of preferred embodiments of the invention, it is to be understood that the disclosure is intended as an illustrative rather than in a limiting sense, as it is contemplated that modifications will readily occur to those skilled in the art, within the spirit of the invention and the scope of the amended claims.

What is claimed is:

1. A compound having a structure analogue to dextro-morphine and dextro-codeine according to formula I: 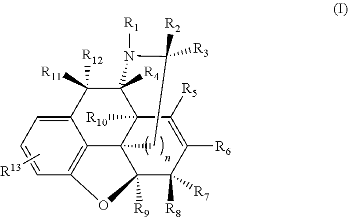
wherein
R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₃ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is 0-4.

2. A compound having a structure analogue to dextro-hydromorphone, dextro-hydrocodone, dextro-oxymorphone and dextro-oxycodone, according to formula II: 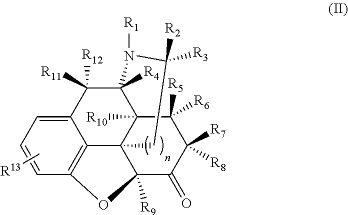
wherein
R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₃ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is 0-4.

3. A compound having a structure analogue to dextro-oripavine and dextro-thebaine, according to formula III: 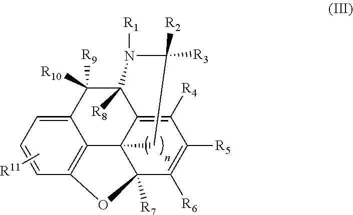
wherein
R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₀ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₀ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₁ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; and
n is 0-4.

4. A compound having a structure analogue to dextro-ethorphine and dextro-buprenorphine, according to formula IV: 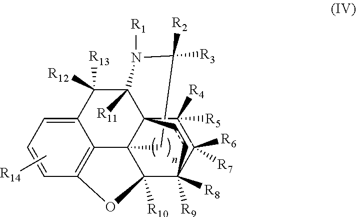
wherein
R₁ is, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₂ to R₁₃ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₂ to R₁₃ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
R₁₄ represents 1-3 substituents selected from the group consisting of hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
n is 0-4; and
the bridged bond between position 9 and 6 of the morphine scaffold is single or double.

5. A method for the treatment or prevention of symptoms, conditions and diseases that may benefit from NMDAR modulation, the method comprising administering a compound being dextromorphine to a subject experiencing a symptom, condition, or disease that may benefit from NMDAR modulation, except for the indications of pain and addiction.

6. The method of claim 5, wherein the compound is formulated as a modified release, long-acting preparation.

7. The method of claim 5, wherein the administering of the compound is performed orally, buccally, sublingually, rectally, vaginally, nasally, via aerosol, transdermally, parenterally, epidurally, intrathecally, intra-auricularly, intraocularly, or topically, including eye drops and other ophthalmic formulations, and including iontophoresis and dermatologic formulations.

8. The method of claim 5, wherein administration of the compound occurs in combination with administering an antidepressant.

9. The method of claim 8, wherein the antidepressant is chosen from an SSRI, an SNERI, and bupropion.

10. The method of claim 5, wherein administration of the compound occurs in combination with administering an antipsychotic.

11. The method of claim 5, wherein administration of the compound occurs in combination with administering a drug that modulates a serotonin receptor.

12. The method of claim 11, wherein the drug that modulates a serotonin receptor is a 5-HT2A agonist.

13. The method of claim 5, wherein administration of the compound occurs in combination with administering magnesium or zinc.

14. A method for the treatment or prevention of symptoms, conditions and diseases that may benefit from NMDAR modulation, the method comprising administering a compound chosen from dextrocodeine, dextrohydromorphone, dextrohydrocodone, dextrooxymorphone, dextrooxycodone, dextrooripavine, dextrothebaine, dextroethorphine, and dextrobuprenorphine to a subject experiencing a symptom, condition, or disease that may benefit from NMDAR modulation.

15. The method of claim 14, wherein the compound is formulated as a modified release, long-acting preparation.

16. The method of claim 14, wherein the administering of the compound is performed orally, buccally, sublingually, rectally, vaginally, nasally, via aerosol, transdermally, parenterally, epidurally, intrathecally, intra-auricularly, intraocularly, or topically, including eye drops and other ophthalmic formulations, and including iontophoresis and dermatologic formulations.

17. The method of claim 14, wherein administration of the compound occurs in combination with administering an antidepressant.

18. The method of claim 17, wherein the antidepressant is chosen from an SSRI, an SNERI, and bupropion.

19. The method of claim 14, wherein administration of the compound occurs in combination with administering an antipsychotic.

20. The method of claim 14, wherein administration of the compound occurs in combination with administering a drug that modulates a serotonin receptor.

21. The method of claim 20, wherein the drug that modulates a serotonin receptor is a 5-HT2A agonist.

22. The method of claim 14, wherein administration of the compound occurs in combination with administering magnesium or zinc.

23. A method for the treatment or prevention of symptoms, conditions and diseases that may benefit from NMDAR modulation, the method comprising administering the compound of claim 1 to a subject experiencing a symptom, condition, or disease that may benefit from NMDAR modulation, wherein the compound is a dextromorphine derivative, a dextrocodeine derivative, a stereoisomer of a dextromorphine derivative or a stereoisomer of a dextrocodeine derivative.

24. The method of claim 23, wherein the compound is formulated as a modified release, long-acting preparation.

25. The method of claim 23, wherein the administering of the compound is performed orally, buccally, sublingually, rectally, vaginally, nasally, via aerosol, transdermally, parenterally, epidurally, intrathecally, intra-auricularly, intraocularly, or topically, including eye drops and other ophthalmic formulations, and including iontophoresis and dermatologic formulations.

26. The method of claim 23, wherein administration of the compound occurs in combination with administering an antidepressant.

27. The method of claim 26, wherein the antidepressant is chosen from an SSRI, an SNERI, and bupropion.

28. The method of claim 23, wherein administration of the compound occurs in combination with administering an antipsychotic.

29. The method of claim 23, wherein administration of the compound occurs in combination with administering a drug that modulates a serotonin receptor.

30. The method of claim 29, wherein the drug that modulates a serotonin receptor is a 5-HT2A agonist.

31. The method of claim 23, wherein administration of the compound occurs in combination with administering magnesium or zinc.

32. A method for the treatment or prevention of symptoms, conditions and diseases that may benefit from NMDAR modulation, the method comprising administering the compound of claim 2 to a subject experiencing a symptom, condition, or disease that may benefit from NMDAR modulation, wherein the compound is a dextrohydromorphone derivative, a dextrohydrocodone derivative, a dextrooxymorphone derivative, a dextrooxycodone derivative, a stereoisomer of a dextrohydromorphone derivative, a stereoisomer of a dextrohydrocodone derivative, a stereoisomer of a dextrooxymorphone derivative, or a stereoisomer of a dextrooxycodone derivative.

33. The method of claim 32, wherein the compound is formulated as a modified release, long-acting preparation.

34. The method of claim 32, wherein the administering of the compound is performed orally, buccally, sublingually, rectally, vaginally, nasally, via aerosol, transdermally, parenterally, epidurally, intrathecally, intra-auricularly, intraocularly, or topically, including eye drops and other ophthalmic formulations, and including iontophoresis and dermatologic formulations.

35. The method of claim 32, wherein administration of the compound occurs in combination with administering an antidepressant.

36. The method of claim 35, wherein the antidepressant is chosen from an SSRI, an SNERI, and bupropion.

37. The method of claim 32, wherein administration of the compound occurs in combination with administering an antipsychotic.

38. The method of claim 32, wherein administration of the compound occurs in combination with administering a drug that modulates a serotonin receptor.

39. The method of claim 38, wherein the drug that modulates a serotonin receptor is a 5-HT2A agonist.

40. The method of claim 32, wherein administration of the compound occurs in combination with administering magnesium or zinc.

41. A method for the treatment or prevention of symptoms, conditions and diseases that may benefit from NMDAR modulation, the method comprising administering the compound of claim 3 to a subject experiencing a symptom, condition, or disease that may benefit from NMDAR modulation, wherein the compound is a dextrooripavine derivative, a dextrothebaine derivative, a stereoisomer of a dextrooripavine derivative or a stereoisomer of a dextrothebaine derivative.

42. The method of claim 41, wherein the compound is formulated as a modified release, long-acting preparation.

43. The method of claim 41, wherein the administering of the compound is performed orally, buccally, sublingually, rectally, vaginally, nasally, via aerosol, transdermally, parenterally, epidurally, intrathecally, intra-auricularly, intraocularly, or topically, including eye drops and other ophthalmic formulations, and including iontophoresis and dermatologic formulations.

44. The method of claim 41, wherein administration of the compound occurs in combination with administering an antidepressant.

45. The method of claim 44, wherein the antidepressant is chosen from an SSRI, an SNERI, and bupropion.

46. The method of claim 41, wherein administration of the compound occurs in combination with administering an antipsychotic.

47. The method of claim 41, wherein administration of the compound occurs in combination with administering a drug that modulates a serotonin receptor.

48. The method of claim 47, wherein the drug that modulates a serotonin receptor is a 5-HT2A agonist.

49. The method of claim 41, wherein administration of the compound occurs in combination with administering magnesium or zinc.

50. A method for the treatment or prevention of symptoms, conditions and diseases that may benefit from NMDAR modulation, the method comprising administering the compound of claim 4 to a subject experiencing a symptom, condition, or disease that may benefit from NMDAR modulation, except for the indications of pain and addiction, wherein the compound is a dextroethorphine derivative, a dextrobuprenorphine derivative, a stereoisomer of a dextroethorphine derivative or a stereoisomer of a dextrobuprenorphine derivative.

51. The method of claim 50, wherein the compound is formulated as a modified release, long-acting preparation.

52. The method of claim 50, wherein the administering of the compound is performed orally, buccally, sublingually, rectally, vaginally, nasally, via aerosol, transdermally, parenterally, epidurally, intrathecally, intra-auricularly, intraocularly, or topically, including eye drops and other ophthalmic formulations, and including iontophoresis and dermatologic formulations.

53. The method of claim 50, wherein administration of the compound occurs in combination with administering an antidepressant.

54. The method of claim 53, wherein the antidepressant is chosen from an SSRI, an SNERI, and bupropion.

55. The method of claim 50, wherein administration of the compound occurs in combination with administering an antipsychotic.

56. The method of claim 50, wherein administration of the compound occurs in combination with administering a drug that modulates a serotonin receptor.

57. The method of claim 56, wherein the drug that modulates a serotonin receptor is a 5-HT2A agonist.

58. The method of claim 50, wherein administration of the compound occurs in combination with administering magnesium or zinc.

59. A method for the treatment or prevention of pain, the method comprising administering a mixture of compounds to a subject experiencing pain or predisposed to experience pain, the compounds including levomorphine and dextromorphine.
